(19) United States
(12) Patent Application Publication
(10) Pub. No.: US 2025/0325419 A1
(43) Pub. Date: Oct. 23, 2025

(54) PEDIATRIC EMERGENCY KIT AND METHODS OF USE THEREOF

(71) Applicant: The Children’s Medical Center Corporation, Boston, MA (US)

(72) Inventor: Peter Ngo, Newton, MA (US)

(21) Appl. No.: 19/037,541

(22) Filed: Jan. 27, 2025

Related U.S. Application Data
(60) Provisional application No. 63/636,596, filed on Apr. 19, 2024.

(51) Int. Cl. A61F 17/00; G06K 19/06

(52) U.S. Cl. CPC A61F 17/00; G06K 19/06037

(57) ABSTRACT

Disclosed is a pediatric emergency kit for esophageal tamponade procedures in pediatric patients and methods of use thereof. The pediatric emergency kit includes at least one esophageal balloon catheter for the tamponade of blood flow, and an inflation hand pump equipped with a pressure gauge for inflating a balloon on the balloon catheter. The pediatric emergency kit can be configured to include multiple compartments, optionally, with a first container/compartment containing at least the esophageal balloon catheter and a second container/compartment containing at least the inflation handpump, and optionally a second esophageal balloon catheter. Optionally, both the first and second containers/compartments contain a syringe, a 3-way luer lock stopcock, and/or a guidewire. Also provided are instructions and/or methods of using the kit for correct placement and inflation of the esophageal balloon to effectively manage arterio-esophageal fistulas.

### CROSS-REFERENCE TO RELATED APPLICATIONS

This application claims the benefit of and priority of U.S. Provisional Application No. 63/636,596, entitled “Pediatric Emergency Kit And Methods Of Use Thereof”, filed in the United States Patent and Trademark Office on Apr. 19, 2024, which is incorporated in its entirety by reference herein.

### FIELD OF THE INVENTION

The disclosed invention is generally in the field of medical kits, particularly for emergency use.

### BACKGROUND OF THE INVENTION

Button battery ingestions result in significant morbidity and mortality in children—before, during, and even after removal. Thousands of children ingest button batteries every year. This increasing trend can be attributed to the expanding utilization of button batteries in electronic devices. Children less than 4 years of age are particularly susceptible to injuries because of their smaller and narrower esophagus, coupled with their tendency to frequently place objects in their mouths.

The injuries created by a button battery lodged in the esophagus develop due to the current of the button battery conducting through saliva and the tissue. When button batteries come into contact with saliva or other bodily fluids, they generate a current, which in turn produces small amounts of corrosive alkaline chemicals such as sodium hydroxide or lye which is also found in drain cleaners and other household products. Lye can burn through the soft tissue of the esophagus, airway and aorta or other large blood vessels, leading to permanent difficulty eating and drinking, infection, and even death. This produces a highly alkaline injury, leading to liquefactive tissue necrosis. Coagulative necrosis of the esophagus can begin about 10-20 minutes after button battery ingestion, and major corrosive injury can occur within hours of ingestion (Tanaka, Esophageal electrochemical burns due to button type lithium batteries in dogs, Vet Hum Toxicol. 1998; 40:193-196). The management of button battery ingestion is particularly challenging since it is associated with complications and a high rate of morbidity and mortality even days after removal of the button battery as the injury progresses through the tissues.

One of the rare, but highly lethal complications of button battery ingestion and impaction in the esophagus is arterio-esophageal fistula (AEF). Arterio-esophageal fistula can occur at all levels of the esophagus, including fistula in the upper-esophagus with the carotid arteries, the mid-esophagus with the aorta, subclavian, and thyroid arteries and lower-esophagus with the aorta. Ingestion of sharp objects can also rarely cause mechanical injuries that result in arterio-esophageal fistula. Arterio-esophageal fistulas that result from button battery ingestion are often fatal even when they occur in a medical setting after battery removal due to the rapidity and large volume of blood loss and the length of time it takes to surgically control bleeding at the site of injury. Given the rarity of arterio-esophageal fistulas in children, most medical providers who encounter these injuries have no prior clinical experience with these injuries and are poorly prepared to rapidly and effectively manage them which further contributes to the poor outcomes seen with these injuries. Therefore, there is a critical need for more effective approaches for controlling bleeding in a child or toddler who has ingested a foreign object, such as a button battery.

It is an object of the invention to provide a pediatric emergency kit for use in an esophageal tamponade procedure in a pediatric patient.

It is a further object of the invention to provide an improved method of creating an esophageal tamponade in a pediatric patient.

### SUMMARY OF THE INVENTION

Pediatric emergency kits for esophageal tamponade procedures in pediatric patients and methods of use thereof are described herein. The pediatric emergency kit includes at least one esophageal balloon catheter for the tamponade of blood flow, and an inflation hand pump equipped with a pressure gauge for inflating a balloon on the balloon catheter. The pediatric emergency kit can be configured to include multiple compartments, optionally, with a first container/compartment containing at least the esophageal balloon catheter and a second container/compartment containing at least the inflation handpump, and optionally a second esophageal balloon catheter. Optionally, both the first and second containers/compartments contain a syringe, a 3-way luer lock stopcock, and a guidewire. Optionally the kit includes instructions for using one or more of the components in the kit. The instructions direct an emergency room medical practitioner to properly place and inflate the esophageal balloon to effectively manage arterio-esophageal fistulas.

### BRIEF DESCRIPTION OF THE DRAWINGS

The accompanying drawings illustrate several embodiments of the disclosed kit and together with the description, serve to explain the principles of the disclosed kit and methods of using the kit.

FIG. 1 shows components of an exemplary pediatric emergency kit.

FIGS. 2A and 2B show an exemplary arrangement of a first, second, and third container in a pediatric emergency kit.

FIG. 3 shows an exemplary pediatric emergency kit in a closed configuration.

FIGS. 4A-4C shows an exemplary esophageal balloon catheter. FIG. 4A shows an exemplary esophageal balloon catheter and an exemplary guidewire. FIGS. 4B and 4C are zoomed in views of the proximal and distal ends of an exemplary esophageal balloon catheter.

FIGS. 5A and 5B show an exemplary inflation hand pump. FIG. 5B is the same as FIG. 5A, and includes an exemplary pressure relief valve 220 that is set to open at or above a set pressure to prevent over-pressurizing the balloon.

FIG. 6 shows an exemplary inflation hand pump connected to an exemplary esophageal balloon.

FIG. 7 shows an exemplary color-coded pressure gauge.

### DETAILED DESCRIPTION OF THE INVENTION

### I. Pediatric Emergency Kit

Pediatric emergency kits for use when treating pediatric esophageal vascular fistulas, e.g. pediatric esophageal arterial fistulas, are disclosed herein along with methods of use thereof. The pediatric emergency kit contains two or more components for controlling blood flow in a child or toddler who has ingested a foreign object such as a button battery.

The pediatric emergency kit generally includes an esophageal balloon catheter for tamponade treatment of an esophageal vascular fistula in a patient, and an inflation hand pump for inflating a balloon on the esophageal balloon catheter. Optionally, the pediatric emergency kit also contains a syringe and a 3-way stopcock, which is suitable for use as an alternative to the inflation hand pump. Optionally, the pediatric emergency kit also includes a guidewire, configured to fit inside the esophageal balloon catheter and guide the advancement of the esophageal balloon catheter through the esophageal lumen of the patient. Optionally, the pediatric emergency kit includes instructions for using one or more of the components in the kit.

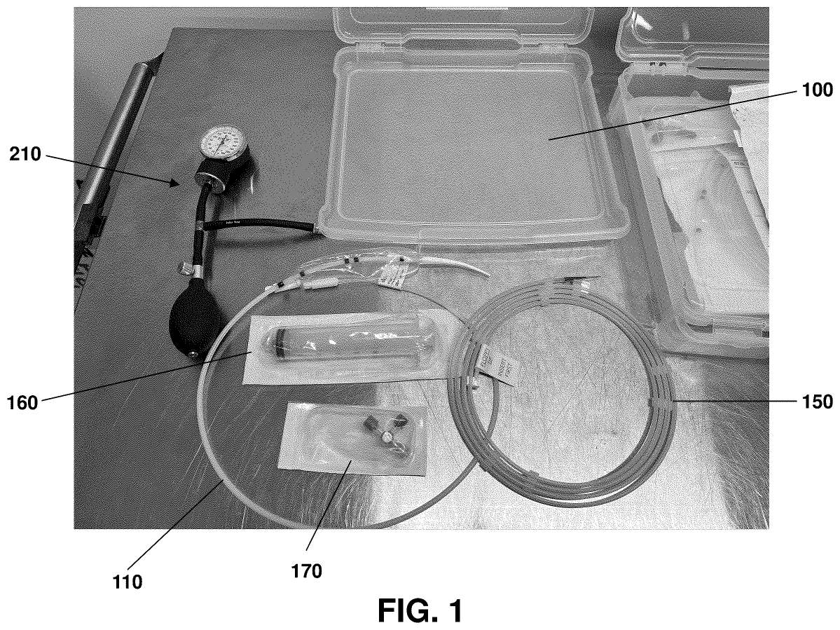

Components of an exemplary pediatric emergency kit are depicted in FIG. 1. As shown in FIG. 1, the kit can contain an esophageal balloon catheter 110 and an inflation hand pump 210; and optionally also includes a guidewire 150, and/or a syringe 160, e.g., a 60 cc syringe, and a 3-way luer stopcock valve 170.

The pediatric emergency kit includes at least a first container or compartment. The first container or compartment is configured for a single use in an emergency setting and is sealed and/or locked, optionally via a breakaway tab. After opening the first container or compartment, the seal and/or breakaway tab is broken, indicating to future users that the container or compartment was previously used and is therefore unsafe for treatment of a new patient. The broken seal or broken tab also indicates that the components of the first container need to be replenished. After replenishing, a new seal or new breakaway tab can be placed on the first compartment or container.

In some forms, the first container or compartment contains at least the esophageal balloon catheter. Optionally, the first container and/or compartment also contains a first guidewire, and/or a first syringe, and a first 3-way luer stopcock. Optionally, the first container also contains a second inflation hand pump. Optionally, the first container contains a further container or bag that contains one or more additional unused breakaway tabs for re-sealing the first container after the components of the first container are replenished after use.

Optionally, the pediatric emergency kit also contains a second container or compartment. The second container or compartment contains an inflation hand pump, and optionally, one or more of the same components that are included in the first container or compartment. For example, the second container or compartment contains at least the inflation hand pump, and a second esophageal balloon catheter. Optionally, the second compartment also contains a second syringe, a second 3-way luer stopcock, and/or a second guide wire. Optionally, the second container is configured to be opened and closed more than one time for reuse. For example, the second container can serve as a demonstration kit, which the user, for example a health care provider, optionally a pediatric doctor or nurse, can open the second compartment or container and practice using the components therein before use in an emergency setting.

Alternatively, the first container can be a re-usable container, configured to be opened and closed more than one time, and the second container can be a single-use container. For example, the first container can serve as the re-usable demonstration kit while the second container can serve as the single-use kit for use in an emergency situation. Optionally, after use, the first and/or second container is replenished with a new esophageal balloon catheter. Optionally, after use, the first and/or second container is also replenished with a new guidewire, and/or a new syringe/or, e.g., a 60 cc syringe, and a new 3-way luer stopcock valve. After replenishing, the first (or second) container can be resealed.

Optionally, the first and second containers or compartments are enclosed in a third container. Optionally, the third container is configured to be opened and closed for more than one use. For example, a user, for example a health care provider, optionally a pediatric doctor or nurse, can open the third container a first time to extract the second container for practice use, and then return the components to the second compartment and/or replace one or more components in the second compartment so that it contains the same components as when it was initially accessed and close the second compartment within the third compartment or container. Subsequently, the same or a different user can open the third container to extract the first container or compartment for use in an emergency e.g., when needed to control blood flow in a pediatric patient.

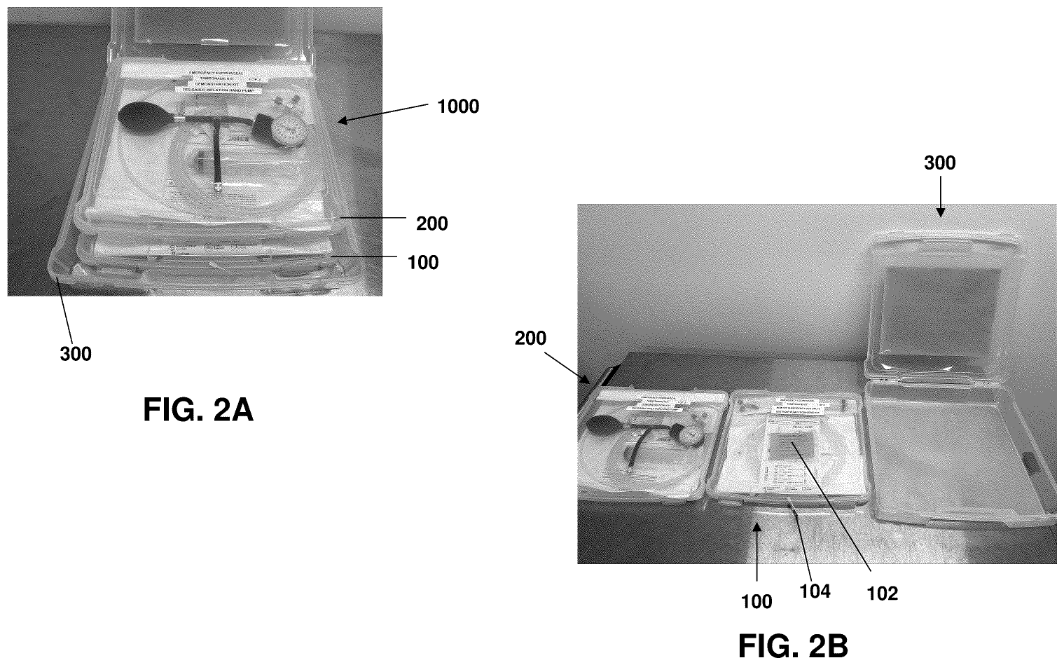

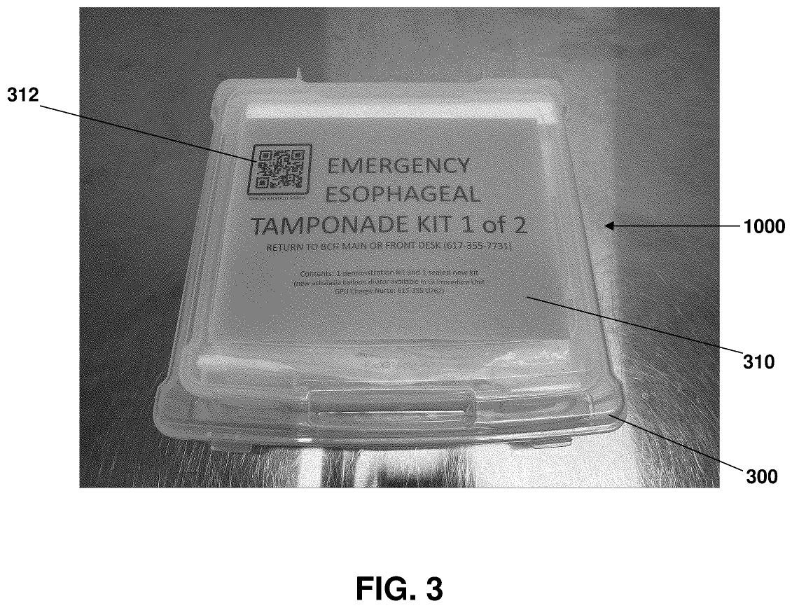

An exemplary pediatric emergency kit is depicted in FIGS. 2A and 2B. The pediatric emergency kit 1000 contains a first container 100 and a second container 200 each of which have a suitable size and shape to both fit inside in a third container 300, when the third container is in a closed position. As shown in FIG. 2B, the first container 100 is a single use kit configured for use in an emergency situation to tamponade blood flow in a pediatric patient. The second container 200 is a reusable demonstration kit that can be used by a medical practitioner, such as a doctor or nurse, for practicing prior to an emergency. The first container 100 can be sealed and/or locked, such as via a breakaway tab 104 (FIG. 2B).

Optionally, the first and second containers or compartments each contain a label describing the contents or components within the first and second containers or compartments. For example, the first container i.e., the container for emergency use optionally contains a visible label such as a card e.g., a colored card, that is attached on the inside of the cover of the first container which lists the contents of the first container and optionally with one or more dates, such as the date the container was sealed and/or the expiration date for the components therein, or spaces for the seal date and/or expiration date to be included. Optionally, a pocket is attached on the inside of the cover of the first container. The pocket can be configured to house multiple copies of the label e.g., multiple cards to facilitate relabeling the container when its contents are replenished. Thus, after the first container is opened and items are replaced when used or expired, the new labels can be already available. For example, as shown in FIG. 2B, the first container 100 has a label or insert 102 listing contents of the first container 100, and optionally the expiration date of the esophageal balloon catheter contained therein.

Optionally, the third container in which the first and second containers or compartments are housed has a label or insert describing its contents. For example, as shown in FIG. 3, the third container 300 of the pediatric emergency kit 1000 has a card, insert or label 310 providing identifying information, such as the name, number or other identifier for the pediatric emergency kit, and the contents therein, such as one demonstration kit and one sealed new kit.

Alternatively, the pediatric emergency kit has a single container or compartment. In this configuration, the esophageal balloon catheter, and the inflation hand pump, and optionally, the syringe, 3-way luer stopcock, and/or the first guidewire are contained in the single container or compartment, optionally along with one or more additional components, instructions, and/or labels.

### A. Esophageal Balloon Catheter

The pediatric emergency kit contains at least one esophageal balloon catheter. Optionally, the pediatric emergency kit contains two esophageal balloon catheters. For example, in a pediatric emergency kit containing a first and a second container or compartment, a first esophageal balloon catheter is in the first container or compartment and a second esophageal balloon catheter is in the second container or compartment.

A suitable esophageal balloon catheter is generally formed of a catheter having at least two ports, and at least one inflatable balloon. Suitable esophageal balloon catheters are commercially available and include but are not limited to RIGIFLEX™ II Achalasia Balloon Dilator (Boston Scientific). Other suitable esophageal balloon catheters can be obtained from Cook Medical (Reference Part #WCAB-30), Hobbs Medical, Inc. (Catalog #3302), and/or Olympus Europe (Article #WA95090A).

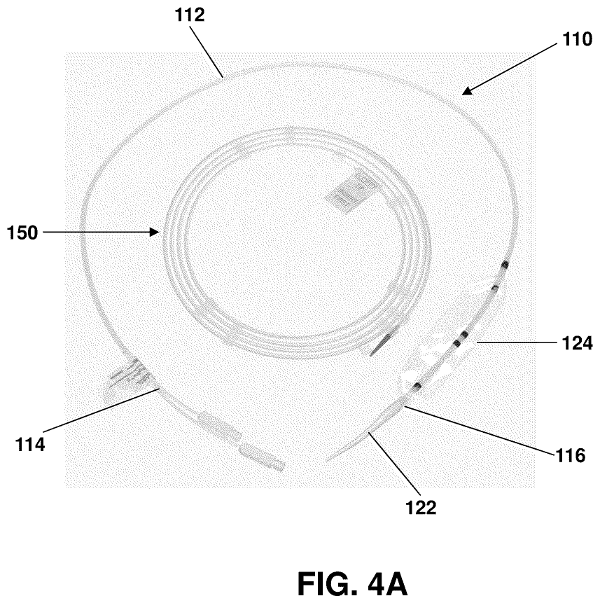

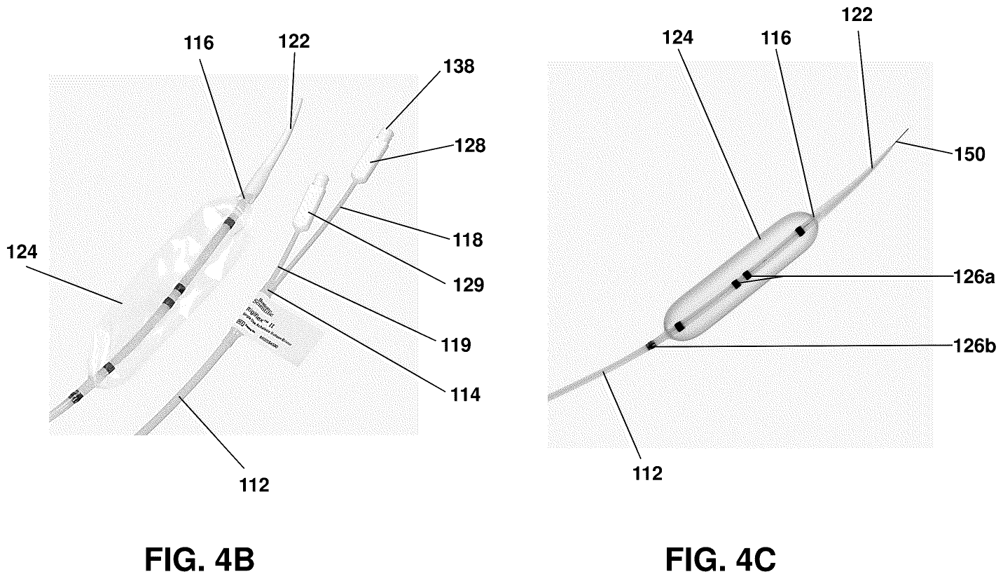

An exemplary esophageal balloon catheter 110 that can be included in the pediatric emergency kit is the RIGIFLEX™ II Achalasia Balloon Dilator (Boston Scientific) is depicted in FIGS. 1 and 4A.

### 1. Catheter

The catheter of the esophageal balloon catheter has a longitudinal lumen having a passage therein, thereby forming a shaft. The shaft has a proximal end and a distal end. The proximal end of the shaft is bifurcated into a first portion and a second portion. The first portion of the proximal end is in fluid communication to a first port and the second portion of the proximal end is fluid communication to a second port. The distal end of the shaft is attached to a flexible distal tip which helps facilitate advancement of the catheter through the esophagus. Optionally, the distal end of the catheter and the flexible distal tip each have openings of suitable size and shape for a guidewire to fit therein.

The first port of the catheter is also in fluid communication with a balloon at the distal end of the catheter and serves as an inflation port. The first port is configured with a stepped end or a Luer lock configured to receive the distal end of a tube of an inflation hand pump or syringe for inflating the balloon.

The second port of the catheter is in fluid communication with the distal end of the catheter and functions as a guide port, through which the guidewire can be removed following insertion of the catheter.

Optionally, the catheter contains a third port in fluid communication with the proximal end of the catheter and with one or more additional ports on the distal end of the catheter. Optionally, one or more additional ports on the distal end of the catheter is positioned between the distal end of the balloon and the flexible distal tip of the catheter. The third port is configured to receive the tip of a syringe or a connector that connects the tip of the syringe or another device to the third port. Optionally, a syringe can be attached to the third port for aspiration, particularly if blood is accumulating beneath the inflated balloon. Optionally, saline can be instilled through the third port to aid in removal of blood from the area.

An exemplary catheter 110 of the esophageal balloon catheter is depicted in FIGS. 4A-4C. Catheter 110 has a longitudinal lumen forming shaft 112. Shaft 112 has a proximal end 114 and a distal end 116 (FIG. 4A). The distal end 116 of shaft 112 is in fluid connection with a flexible distal tip 122. The proximal end 114 bifurcates into a first portion 118 which is connected to a first port 128, and a second portion 119 which is connected to a second port 129 (FIG. 4B). The first port 128 of catheter 110 functions as an inflation port and is in fluid connection with a balloon 124, and the second port 129 is in fluid communication with the distal end 116 of catheter 110 and functions as a guide port (FIGS. 4B and 4C). As shown in FIG. 4B, the first port 128 also has a stepped end 138 configured to be connected to an inflation hand pump. As shown in FIG. 4C, shaft 112 of catheter 110 contains an opening suitable for guiding the catheter 110 over a guidewire 150.

i. Dimensions

The shaft of the catheter has suitable length and diameter to be inserted into and run along the length of a constricted space within the esophagus. Optionally, the catheter has a length of about 60 cm to about 90 cm. Optionally, the catheter has a length of about 60 cm to about 90 cm, about 60 cm to about 85 cm, about 60 cm to about 70 cm, about 60 cm to about 75 cm, or about 60 cm to about 80 cm.

Optionally, the shaft of the catheter has a diameter of at least about 0.35 cm to about 0.50 cm. In some forms, the shaft of the catheter has a diameter of at least about 0.36 cm to about 0.50 cm, about 0.38 cm to about 0.50 cm, about 0.40 cm to about 0.50 cm, 0.42 cm to about 0.50 cm, about 0.44 cm to about 0.50 cm, 0.46 cm to about 0.50 cm, or about 0.48 cm to about 0.50 cm.

ii. Materials

The catheter of the esophageal balloon catheter can be made of any suitable medical grade materials for making catheters including but not limited to silicone rubber, latex rubber, polyvinyl chloride (PVC), and polyethylene. Optionally, the catheter of the esophageal balloon catheter is made of medical grade PVC. Medical grade materials include materials that have been tested and certified to operate safely near patients.

iii. Markings

The shaft of the catheter has an outer surface which can include two or more radiopaque markings to facilitate visualization of the catheter when in use e.g., during a procedure.

Optionally, a portion of the radiopaque markings are located in the region of the catheter located within the balloon to provide visualization of the balloon during a procedure. For example, as shown in FIG. 4C, the outer surface of shaft 112 of the catheter has a plurality of radiopaque markings 126a in the region of the catheter located within balloon 124.

Optionally, one or more radiopaque markings are positioned on the outer surface of the catheter adjacent to both ends of the balloon, optionally in the regions of the catheter that align with both ends of the balloon. For example, as shown in FIG. 4C, shaft 112 contains a radiopaque marking 126b in the region of the catheter that aligns with one end of the balloon 124.

Optionally, the catheter includes markings indicating the distance that the balloon is inserted into the patient, relative to the patient's incisor teeth. Optionally, the markings indicating measurements start about 5 cm to about 10 cm above the proximal end of the balloon and continue towards the proximal end of the catheter until about 30 cm, about 35 cm, about 40 cm, until about 45 cm, measured from the midpoint of the balloon. The markings can be spaced at regular intervals, such as about 0.5 cm apart, about 1 cm apart, about 2 cm apart, about 3 cm apart, about 4 cm apart, or about 5 cm apart. Optionally, the markings include numeric indications and/or lines.

### 2. Balloon

The esophageal balloon catheter contains an inflatable balloon positioned around the shaft of the catheter towards the distal end of the catheter near the distal flexible tip. The balloon is in fluid connection with the first port at the proximal end of the catheter.

The balloon can be inflated and deflated as needed to occlude blood flow within the esophageal lumen. For example, during a procedure, the balloon is deflated, i.e., all the air in the balloon is removed, prior to insertion of the esophageal balloon catheter into the esophagus of the patient. Once the esophageal balloon catheter is fully inserted into the esophagus, e.g., near a vascular fistula in a toddler, the balloon can be inflated by connecting and applying pressure to an inflation hand pump (described below) to the first port of the esophageal balloon catheter. It is important not to exceed the maximum inflation pressure specified for the balloon as surpassing this pressure could lead to balloon rupture. Optionally, the balloon can be lubricated prior to insertion into the patient's esophagus.

An exemplary balloon 124 of an esophageal balloon catheter 110 is depicted in FIGS. 4A-4C. As shown in FIGS. 4A and 4B, balloon 124 is positioned around shaft 112 near the distal end 116 of catheter 110. Balloon 124 is deflated prior to insertion of the esophageal balloon catheter 110 in the esophagus of a patient (FIG. 4B) and inflated when the esophageal balloon catheter 110 is in use after insertion of the esophageal balloon catheter to the appropriate position in the esophageal lumen of the patient (FIG. 4C).

i. Dimensions

The balloon on the esophageal balloon catheter has a size and diameter that is compatible with the diameter of the esophageal lumen of a child or toddler. In some forms, the esophageal balloon has a diameter in its widest region when fully inflated of about 20 to about 40 mm, about 25 mm to about 40 mm, about 30 to about 40 mm, about 35 to about 40 mm, about 25 mm to about 35 mm, about 25 mm to about 32 mm, about 25 mm to about 30 mm, or about 25 mm to about 28 mm. Optionally, the balloon has a diameter when fully inflated of about 25 mm, about 30 mm, about 35 mm.

The balloon has a rounded proximal end and a rounded distal end connected by a central section having a uniform diameter. In some forms, the central section of the balloon has a length of about 5 cm to about 15 cm, about 8 cm to about 15 cm, or about 11 cm to about 15 cm. Optionally the central section of the balloon has a length of about 10 cm.

ii. Materials

The balloon of the esophageal balloon catheter can be formed of any suitable flexible medical grade materials for making balloons including but not limited to polyesters such as poly(ethylene terephthalate) (PET), polyurethane, Pebax, silicone, crosslinked polyethylene, polyethylene terephthalate, polyolefin copolymer, rubber, or nylon. Optionally, the balloon can be formed of polyurethane or Pebax.

### B. Inflation Hand Pump

The pediatric emergency kit also includes at least one inflation hand pump for inflation of the balloon when the esophageal balloon catheter is in use. Optionally, the inflation hand pump is contained in the second container or compartment of the pediatric emergency kit. Exemplary commercially available inflation hand pumps that can be included in the pediatric emergency kit are manufactured by Accoson, Hammarplast combination hand pump/pressure gauge, Handpump including manometer (Hammarplast Medical, Article #9005, Handpump with manometer (Carumed, Article #314010), Airius® Palm Aneroid Sphygmomanometer (MDF Instruments, Catalog #MDF848AR12), and Handheld Palm Aneroid Sphygmomanometer (Medline, Part #MDSANEROIDHP). However, the commercially available hand pumps listed above do not contain a suitable adapter configured for attachment to one of the ports of the esophageal balloon catheter described above, such as a port on the Rigiflex II achalasia balloon. Commercially available hand pumps can be modified to provide a suitable adapter for attachment to the esophageal balloon catheter. For example, the commercially available hand pumps listed above can be modified by replacing the barbed adapter or palm adapter with a luer-lock connector e.g., a male luer-lock connector. Preferably, the modified connector is not a compression fit barbed connector as this would require the extra step of pressing the two connectors together with enough force to avoid losing pressure, which is not necessary with a standard medical luer-lock connector.

The inflation hand pump has two ends, a first end and a second end in fluid communication with each other. The first end of the inflation hand pump contains an inflation bulb in fluid communication with a pressure gauge. The second end of the inflation hand pump is configured to connect with the first port i.e., an inflation port of the esophageal balloon catheter, via a suitable connector, such as a luer lock connector, luer slip connector, or other suitable connector.

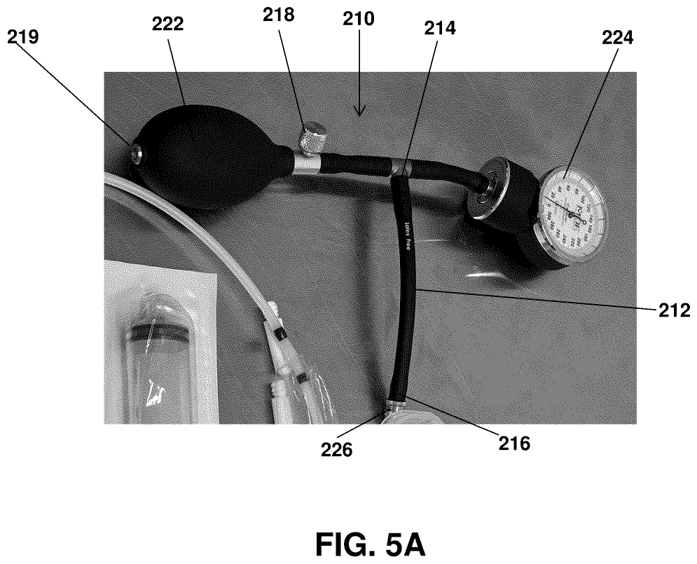

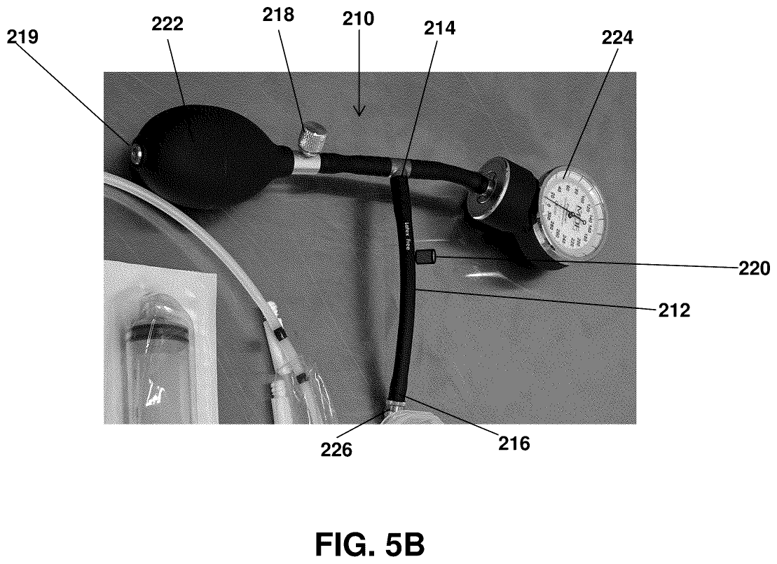

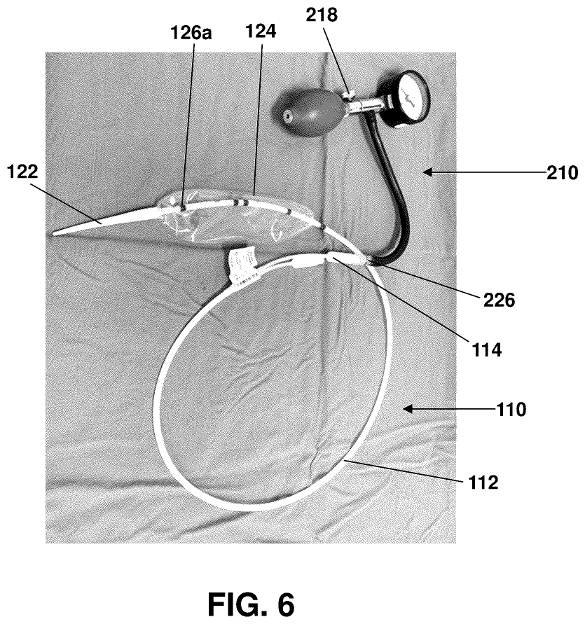

An exemplary inflation hand pump is depicted in FIGS. 5A and 5B. As shown in FIG. 5A, the inflation hand pump 210 has a longitudinal tube forming a channel 212 having a first end 214 in fluid communication with a second end 216. The first end 214 contains an inflation bulb 222 in fluid communication with a pressure gauge 224. The second end 216 is connected to a luer lock connector 226, which is configured to connect to the first port of an esophageal balloon catheter (FIGS. 5 and 6). As shown in FIGS. 5A and 5B, inflation bulb 222 contains a screw valve 218 on one end of the inflation bulb or in fluid communication with the inflation bulb and configured to control the flow of air into an esophageal balloon catheter when in use. The opposite end of the bulb includes an air inlet valve 219 that opens when the inflation bulb is squeezed, allowing air to enter the inflation bulb.

Optionally, a pressure relief valve that automatically opens when the pressure in the balloon reaches a set pressure to prevent a user from accidentally over-pressurizing the balloon when the esophageal balloon catheter is in use. For example, the set pressure at which the pressure relief valve is set to automatically open is in the range of about 150 mmHg to 200 mmHg, in the range of about 150 mmHg to 160 mmHg, or in the range of about 150 mmHg to 180 mmHg. For example, the pressure in the balloon at which the pressure relief valve is set to automatically open is greater than about 150 mmHg, or is about 155 mmHg or greater, about 160 mmHg or greater, about 165 mmHg or greater, about 170 mmHg or greater, about 175 mmHg or greater, about 180 mmHg or greater, about 185 mmHg or greater, about 190 mmHg or greater, about 195 mmHg or greater, or about 200 mmHg.

The pressure relief valve can be located on or integrated into the inflation bulb. Optionally, the pressure relief valve is integrated into the screw valve so that the screw valve automatically opens when a set pressure is reached or exceeded, as described above. Alternatively, the pressure relief valve is in fluid communication with the outlet of the inflation bulb and is located after the outlet of the inflation bulb, but before the screw valve or is located between the screw valve and the balloon. For example, as shown in FIG. 5B, the pressure relief valve 220 is in fluid communication with the outlet of the inflation bulb 222 and is located between the screw valve 218 and the balloon 124.

### 1. Pressure Gauge

The pressure gauge includes markings to distinguish pressures in the physiologic blood pressure range, optionally wherein the pressure gauge measures pressures in the range of about 20 mmHg to about 300 mmHg. The maximum pressure denoted on the pressure gauge is in the range of about 500 mmHg to about 400 mmHg, about 400 mmHg to about 300 mmHg, about 300 mmHg to about 250 mmHg, about 300 mmHg to about 200 mmHg, about 250 mmHg to about 200 mmHg. or about 200 mmHg.

In some forms, the highest pressure on the pressure gauge is 200 mmHg, 190 mmHg or 180 mmHg.

In some forms, the pressure gauge included on the inflation hand pump may be a color-coded pressure gauge. For example, the color-coded pressure gauge may have two, three or four colors, each color depicting a different pressure range. Alternatively, the color-coded pressure gauge may have more than four colors.

Optionally, a first portion of the pressure gauge dial is a coded with a first color, such as green, and covers a pressure in the range from about 20 mmHg to about 130 mmHg, 30 mmHg to about 130 mmHg, 40 mmHg to about 130 mmHg, 50 mmHg to about 130 mmHg, 60 mmHg to about 130 mmHg, 70 mmHg to about 130 mmHg, or 80 mmHg to about 130 mmHg. Optionally, a second portion of the pressure gauge dial is coded with a second color, such as yellow, and covers a pressure in the range from about 130 mmHg to about 150 mmHg. Optionally, a third portion of the pressure gauge dial is coded with a third color, such as red, and covers a pressure in the range from about 150 mmHg to about 300 mmHg, 150 mmHg to about 200 mmHg, 150 mmHg to about 190 mmHg, or 150 mmHg to about 180 mmHg.

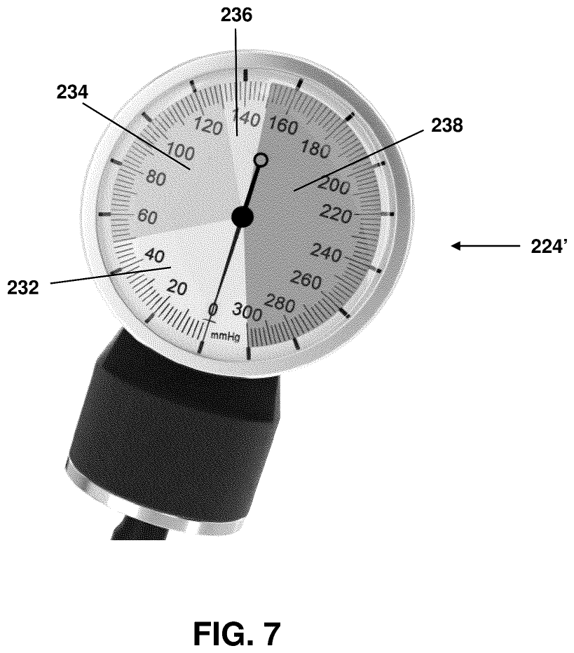

For example, as shown in FIG. 7, the pressure gauge 224′ has four color regions, a white region 232, a green region 234, a yellow region 236, and a red region 238. The green region 234 denotes a pressure range between 50 mmHg to 130 mmHg. When the pressure gauge 224′ indicates a pressure in the green region 234, the user knows that enough pressure is being supplied to inflate the balloon on the esophageal balloon catheter. When the pressure gauge 224′ indicates a pressure in the white region 232 or in the yellow 236 and red 238 regions, not enough or too much pressure respectively, is being supplied to the balloon.

### C. Syringe

Optionally, the pediatric emergency kit additionally includes a 50-70 cc syringe and a 3-way luer lock stopcock as an alternative to the hand pump. The syringe can be used in case the inflation hand pump malfunctions or is missing from the kit.

A first end of the 3-way stopcock is configured for attachment to the tip of the syringe and second end of the 3-way stopcock is configured for attachment to the first port, i.e., the inflation port, of the esophageal balloon catheter. The third end is exposed to the environment.

In use, the distal tip of the syringe may be attached to a first side of the 3-way stopcock and the stopcock is turned to the closed position cutting off the third end. The plunger of the syringe may be pulled back to fill the syringe with air. Then the second end of the stopcock is connected to the first port i.e., the inflation port, of the esophageal balloon catheter.

The stopcock can then be switched to the open position, and the balloon can be deflated e.g., by squeezing the balloon, to remove the air prior to insertion into the esophagus of the patient.

Once the esophageal balloon catheter is inserted into esophagus of the patient at the desired position, the stopcock is switched to the closed position and the user can inflate the balloon on the esophageal balloon catheter by pushing the plunger into the barrel of the syringe.

### D. Guidewire

In some forms, the kit includes a guidewire having a suitable size and shape for the catheter to fit over the guidewire, and to allow the esophageal balloon catheter to be advanced along the guidewire. The guidewire can have a length ranging from about 110 cm to about 180 cm, about 120 cm to about 180 cm, about 130 cm to about 180 cm, about 140 cm to about 180 cm, about 150 cm to about 180 cm, about 150 cm to about 180 cm, or about 160 cm to about 180 cm. The guidewire can have a thickness of less than 0.050 inches. For example, the guidewire may have a thickness of about 0.035 inches to about 0.050 inches, about 0.038 inches to about 0.050 inches, about 0.042 inches to about 0.050 inches, or about 0.045 inches to about 0.050 inches. Optionally, the guidewire has a thickness of about 0.038 inches.

Optionally, the guidewire has a stiff proximal tip and floppy distal tip which aids in facilitating advancement of the guidewire through the esophagus and decreases the risk of injury from the tip of the advanced guidewire. Optionally, the floppy distal tip has a length of about 3 cm to about 5 cm of guidewire.

In use, the floppy distal tip of the guidewire is inserted into the mouth of the patient and through to the stomach to ensure that the guidewire traverses the entire length of the esophagus without entering the airway or crossing the fistula. The catheter can then be advanced over the guidewire by inserting the flexible distal tip of the catheter over the stiff proximal tip of the guidewire and advancing the catheter over the length of the guidewire that is positioned within the esophagus. In some forms, the guidewire can also be inserted into the stomach under direct visualization by advancing the guidewire through the working channel of a videoendoscope then leaving the wire in position while removing the videoendoscope.

Exemplary commercially available guidewires that can be included in the pediatric emergency kit can be obtained from Medline (Catalog Nos. DYNJGWIRE01, DYNJGWIRE02, DYNJGWIRE48), ConMed (Catalog #CNM-025260S), Medtronic (Catalog #N25180), and Boston Scientific (Catalog #: 46-155B, 46-156B, 46-159B, 46-165B, 46-166B). Other commercially available guidewires can be used if they have an appropriate length to be compatible with the length of the catheter, a diameter between about 0.025 inches and about 0.038 inches, and a flexible tip.

### E. Instructions for Use

The pediatric emergency kit also contains instructions for using the components of the kit to tamponade blood flow in a pediatric patient e.g., a toddler. Optionally, one or more of the instructions are provided via a Quick Response (“QR”) code positioned at any suitable position on the kit. For example, as shown in FIG. 3, the QR code 312 can be positioned on the top left side of the third compartment 300 of the pediatric emergency kit 1000. Alternatively, the QR code can be positioned at any suitable position on the first container, the second container, or both the first container and second container.

Scanning the QR code with an electronic device, e.g., a phone or tablet, provides a user with a link to the instructions for using the pediatric emergency kit. Optionally, the link to the instructions provides a list of steps that the user, e.g., a doctor can easily read and replicate. Optionally, the link to the instructions provides a demonstration video that the user can watch and follow.

Alternatively, the instructions for use are provided on a card e.g., index card, paper, or other suitable printed media in the first, second and/or third compartments of the pediatric emergency kit.

Alternatively, the instructions for using the pediatric emergency kit are provided on an electronic device, e.g., a USB drive, an SD card, or a microSD card which can be connected to a computer for accessing the instructions.

### II. Instructions for Using the Pediatric Emergency Kit

Optionally, one or more pediatric emergency kits are stored in a central location, such as in the operating room.

Instructions for Using an Esophageal Balloon Catheter Assembled with an Inflation Hand Pump to Tamponade Blood Flow.
Instructions without a Guidewire

Disclosed are instructions and methods for using the pediatric emergency kit for creating an esophageal tamponade in a pediatric patient in need thereof. In some instances, there is insufficient time to use a guidewire to guide the catheter into its desired location, in such instances the instructions provided in or on the kit can direct the user to practice the following steps:
(a) insert the distal tip of the esophageal balloon catheter through the patient's mouth and advance the tip of the catheter through the patient's esophagus until the balloon is aligned with the location of the arterio-esophageal fistula;
(b) confirm that the balloon of the esophageal balloon catheter is aligned with the location of the arterio-esophageal fistula; and
(c) use the inflation hand pump to inflate the balloon to a pressure that is above systolic pressure, such as about 130 mmHg.

An exemplary esophageal balloon catheter, e.g., an achalasia balloon, fully assembled with an inflation hand pump and ready for use, is depicted in FIG. 6. The first port i.e., the inflation port 114 of the esophageal balloon catheter 110 can be connected to via a luer lock connector 226 of the inflation hand pump 210 (FIG. 6). Although not apparent from FIG. 6, valve 218 on the inflation hand pump 210 must be in the open position to facilitate deflation of the balloon 124 e.g., by squeezing the balloon 124 to remove any excess air. The floppy tip 122 of the esophageal balloon catheter 110 can then be inserted into the patient's mouth and through the esophagus to the approximate location of the suspected vascular fistula formed due to the corrosive chemical from the button battery.

Optionally, the physician or medical professional can use fluoroscopy to guide positioning of the esophageal balloon catheter via the radiopaque markings 126a on the shaft 112 of the esophageal balloon catheter 110 (FIG. 6). Optionally, radiography may be used to determine the location of the foreign object, e.g., a button battery, thereby allowing determination of the position of the vascular fistula in the esophagus. Thus, the distance from the patient's mouth to the required placement of the balloon 124 can be estimated and marked on shaft 112 of the catheter, for example, with a piece of tape or a marker (FIG. 6). This allows the physician to accurately determine how far to insert the esophageal balloon catheter to reach the targeted location. Optionally, markings indicating measurements on the catheter can be used to measure the distance that the balloon is inserted from the patient's incisor teeth. Optionally, the distance from the incisors to the esophageal injury are known from prior measurement, such as with an endoscope. Optionally, the catheter includes markings indicating measurements spaced at regular intervals, such as about 0.5 cm apart, about 1 cm apart, about 2 cm apart, about 3 cm apart, about 4 cm apart, or about 5 cm apart. Optionally, the catheter can have measurements marked at intervals, such as with numeric indications and/or lines. For example, the catheter can have measurements indicated by lines at 1 cm intervals, and numerically printed at every 5 cm. Optionally, markings indicating measurements start approximately 5 cm from the top of the proximal end of the balloon and indicate measurements from the distance to the balloon's midpoint.

Optionally, balloon 124 can be lubricated and compressed to remove any air prior to insertion into the patient's mouth. The flexible distal tip 122 of esophageal balloon catheter 110 can be advanced into the patient's mouth to the expected location of the vascular fistula.

Once in position, the inflation hand pump 210 can be used to inflate balloon 124 by closing the screw valve 218 on the inflation hand pump 210 and squeezing the inflation bulb 222 until the balloon 124 is inflated up to a pressure that is above systolic blood pressure (FIG. 6). In some forms, the pressure at which the balloon inflates is about 130 mmHg, which is considered greater than the systolic blood pressure for a child (the normal systolic blood pressure for a child is in the range of 90 mmHg to about 110 mmHg). However, if sufficient inflation pressure is not used, trauma to the esophagus may result. For example, in some cases, 120 mmHg may be sufficient to apply pressure to the walls of the esophagus to tamponade systolic bleeding, but not enough pressure to fully inflate the balloon to its maximum diameter (e.g. 30 mm for most achalasia balloons), which can result in damage or injury to the patient's esophagus. Optionally, if the patient's blood pressure is being measured at the time of tamponade balloon placement with either a blood pressure cuff or an arterial line blood pressure monitor, then the tamponade balloon can be inflated to a pressure that is about 10 mmHg to about 15 mmHg above the measured systolic blood pressure.

If bleeding from an arterio-esophageal fistula does not stop after inflation of the tamponade balloon, then the provider should reassess the position of the balloon and whether the balloon pressure is greater than the patient's systolic blood pressure.

Instructions with a Guidewire

In some instances, there is sufficient time to use a guidewire to guide the catheter through the patient's esophagus and into its desired location. Thus, prior to performing step (a) of the method described above, the method can further include (a′) inserting a guidewire into the patient's mouth, through the esophagus and into the patient's stomach.

Prior to insertion, the guidewire is removed from the sheath to allow the physician or medical professional to access the floppy end. The floppy end can then be inserted in through the patient's mouth through the esophagus to the stomach to ensure that (i) the guidewire traverses the entire length of the esophagus, and (ii) the guidewire does not enter the patient's airway or cross the fistula.

Optionally, step (a′) includes inserting the proximal end of the guidewire into the opening at the distal end of the catheter and advancing the esophageal balloon dilation catheter over the guidewire until the balloon is aligned with the location of the arterio-esophageal fistula.

Optionally, step (a′) further comprises inserting an endoscope into the patient's mouth through the esophagus and into the patient's stomach to facilitate positioning of the guidewire into the stomach, and then the esophageal balloon catheter may be advanced over the distal end of the guidewire into to the patient's mouth. When the button battery is removed via endoscopy, should bleeding occur, the esophageal balloon catheter may be rapidly advanced over the guidewire across the defect to tamponade the bleeding.

In some forms, when the esophageal balloon catheter contains a third port in fluid communication with the proximal end of the catheter, and one or more additional ports on the side of the distal end of the catheter positioned between the distal end of the balloon and the flexible distal tip of the catheter. In these forms, the method may include one or more of the following steps:
(d) aspirating with a syringe from the third port on the catheter to assess whether there is ongoing blood accumulation below the inflated balloon; if blood accumulation is observed,
(e) subsequently instilling saline through the third port, and then
(f) repeating syringe aspiration through the third port to observe if the accumulated blood has cleared, optionally repeating steps (d)-(f) until the accumulated blood has cleared. In some cases, if the blood does not clear after instilling saline and there is clinical evidence of ongoing bleeding, then it is possible that the balloon of the esophageal balloon catheter has been placed proximal to the arterio-esophageal fistula site, and bleeding is continuing distal to the balloon position. In these cases, the provider can optionally re-assess the position of the balloon using fluoroscopy measurements on the catheter. If the provider wishes to re-position the balloon, the pressure in the balloon can be released e.g. by opening the valve on the hand inflation pump, and the balloon can be re-positioned, and then re-inflated.
   Instructions for Using an Esophageal Balloon Catheter Assembled with a Syringe and a 3-Way Stopcock to Tamponade Blood Flow.

In an alternative configuration, a syringe and a 3-way stopcock can be used to inflate the balloon of the esophageal balloon catheter, e.g., if the inflation hand pump malfunctions or is missing.

An exemplary method for using an esophageal balloon catheter assembled with a syringe and a 3-way stopcock to tamponade blood flow includes one or more of the following steps:
(i) A syringe, e.g., a 50 mL or 60 mL syringe is attached to one end of a 3-way stopcock and filled with air;
(ii) The first port i.e., the inflation port of the esophageal balloon catheter is connected to the 3-way stopcock via the luer lock connector;
(iii) the air port on the 3-way stopcock is opened and the balloon is deflated to its smallest size e.g., by squeezing;
(iv) the esophageal balloon is inserted into the patient's esophagus to the appropriate distance; and
(v) once in position in the esophagus, the air port on the 3-way stopcock is closed and the balloon is inflated to the appropriate pressure by pushing the plunger of the syringe into the barrel of the syringe,
(vi) once the balloon is filled with appropriate pressure to tamponade bleeding the balloon port can be closed with the stopcock, and
(vii) if additional pressure is desired in the balloon then the syringe plunger can be withdrawn while the balloon port is closed and then the stopcock can be moved to close the air position and the plunger can be pushed into the syringe barrel to further increase balloon pressure.

The described kit and methods will be better understood in view of the following numbered paragraphs.
1. A pediatric emergency kit comprising:
   (i) an esophageal balloon catheter, comprising two ports in fluid communication with the proximal end of a catheter, wherein a first port is in fluid communication with the balloon and the second port is in fluid communication with the distal end of the catheter, and a flexible distal tip at the distal end of the catheter; and
   (ii) an inflation hand pump, comprising a pressure gauge, wherein the pressure gauge includes colors and/or markings to distinguish pressures in the physiologic blood pressure range, optionally wherein the pressure gauge measures pressures in the range of 20 mmHg to 300 mmHg.
2. The pediatric emergency kit of paragraph 1, wherein the esophageal balloon catheter has a length of about 60 to about 90 cm.
3. The pediatric emergency kit of paragraph 1 or paragraph 2, wherein the balloon on the esophageal balloon catheter has a diameter in its widest region when fully inflated of about 25 mm to about 30 mm.
4. The pediatric emergency kit of any one of paragraphs 1-3, further comprising a guidewire that has a flexible distal tip, and wherein the guidewire has a suitable size and shape for the esophageal balloon catheter to fit over the guidewire, and to allow the esophageal balloon dilation catheter to be advanced along the guidewire.
5. The pediatric emergency kit of any one of paragraphs 1-4, wherein the esophageal balloon catheter is inside a first container or compartment that is sealed and/or locked, optionally with a break-away tab.
6. The pediatric emergency kit of any one of paragraphs 1-5, further comprising (iii) a 50-70 cc syringe and (iv) a 3-way luer lock stopcock, wherein the 3-way luer stopcock is configured for attachment to the first port of the esophageal balloon catheter.
7. The pediatric emergency kit of paragraphs 5 and 6, wherein the first container or compartment further includes (iii) and (iv) therein.
8. The pediatric emergency kit of any one of paragraphs 5-7, further comprising a second container or compartment, wherein the second container or compartment contains therein (i′) a second esophageal balloon catheter and (ii) the inflation hand pump; optionally the second container or compartment further contains (iii′) a second 50-70 cc syringe, and (iv′) a second 3-way luer lock stopcock.
9. The pediatric emergency kit of paragraph 8, wherein the second container or compartment is configured to be opened and closed more than one time for reuse.
10. The pediatric emergency kit of any one of paragraphs 8 and 9, wherein both the first and second containers or compartments are enclosed in a third container or compartment that is configured to be opened and closed more than one time for reuse.
11. The pediatric emergency kit of any one of paragraphs 1 to 10, further comprising instructions for use, optionally wherein one or more of the instructions are provided via a QR code.
12. The pediatric emergency kit of any one of paragraphs 1-11, wherein the maximum pressure denoted on the pressure gauge is in the range of about 300 mmHg to about 250 mmHg, 300 mmHg to 200 mmHg, about 250 mmHg to about 200 mmHg, or about 200 mmHg.
13. The pediatric emergency kit of any one of paragraphs 1-12, wherein the distal end of the esophageal balloon catheter includes an opening having a suitable size and shape for a guidewire to fit therein.
14. The pediatric emergency kit of any one of paragraphs 1-13, wherein the outer surface of the catheter in the esophageal balloon catheter includes two or more radiopaque markings, optionally wherein at least a portion of the radiopaque markings are located in the region of the catheter located within the balloon.
15. The pediatric emergency kit of any one of paragraphs 1-14, wherein the catheter in the esophageal balloon catheter includes markings, wherein the markings begin about 5 cm to about 10 cm above the proximal end of the balloon and end at a distance of about 30 cm, about 35 cm, about 40 cm, or about 45 cm from the midpoint of the balloon, optionally wherein the markings are spaced at regular intervals.
16. The pediatric emergency kit of any one of paragraphs 1-15, wherein the balloon is formed from a flexible material selected from the group consisting of polyesters such as poly (ethylene terephthalate) (PET), Pebax, silicone, crosslinked polyethelene, polyethylene terephthalate, polyurethane, polyolefin copolymer, rubber, or nylon.
17. The pediatric emergency kit of any one of paragraphs 1-16, further comprising a pressure relief valve configured to open when the pressure in the balloon reaches a set pressure, wherein the pressure relief valve is in in fluid communication with the outlet of the inflation bulb,
optionally the pressure relief valve opens automatically when the pressure in the balloon is in the range of about 150 mmHg to 200 mmHg.
18. The pediatric emergency kit of any one of paragraphs 1-17, wherein the esophageal balloon catheter further comprises a third port in fluid communication with the proximal end of a catheter and with one or more additional ports on the side of the distal end of the catheter positioned between the distal end of the balloon and the flexible distal tip of the catheter.
19. The pediatric emergency kit of any one of paragraphs 1-18, wherein the pressure gauge dial is color coded, optionally wherein a first portion of the pressure gauge dial is a coded with a first color, such as green, and a second portion of the pressure gauge dial is coded with a second color, such as yellow, and a third portion of the pressure gauge dial is coded with a third color, such as red.
20. The pediatric emergency kit of paragraph 19, wherein the first portion of the pressure gauge dial ranges from 50 mmHg to 130 mmHg, optionally wherein the second portion of the pressure gauge dial ranges from 130 mmHg to 150 mmHg.
21. A method of creating an esophageal tamponade in a pediatric patient in need thereof, comprising the steps of:
   (a) inserting the flexible distal tip of the esophageal balloon catheter provided in the kit of any one of paragraphs 1 to 20 through the patient's mouth and advancing the flexible distal tip of the esophageal balloon catheter through the patient's esophagus until the balloon is aligned with the location of the arterio-esophageal fistula;
   (b) confirming that the balloon of the esophageal balloon catheter is aligned with the location of the arterio-esophageal fistula;
   (c) using the handpump provided in the kit of any one of paragraphs 1 to 19, to inflate the balloon to a pressure that is above systolic pressure, such as about 130 mmHg.
22. The method of paragraph 21, further comprising prior to step (a), (a′) inserting a guidewire through the patient's mouth, through the esophagus and into the patient's stomach.
23. The method of paragraph 22, wherein step (a′) further comprises inserting an endoscope through the patient's mouth through the esophagus and into the patient's stomach to facilitate positioning of the guidewire.
24. The method of paragraph 22, wherein step (a) further comprises inserting the proximal end of the guidewire into the opening at the distal end of the catheter and advancing the esophageal balloon dilation catheter over the guidewire until the balloon is aligned with the location of the arterio-esophageal fistula.
25. The method of any one of paragraphs 21-24, wherein the esophageal balloon catheter further comprises a third port in fluid communication with the proximal end of a catheter and with one or more additional ports on the side of the distal end of the catheter positioned between the distal end of the balloon and the flexible distal tip of the catheter, further comprising:
   (d) aspirating with a syringe from the third port on the catheter to assess whether there is ongoing blood accumulation below the inflated balloon; and
   if blood accumulation is observed:
   (e) subsequently instilling saline through the third port,
   (f) repeating syringe aspiration through the third port to observe if the accumulated blood has cleared, optionally repeating steps (d)-(f) until the accumulated blood has cleared, and
   (g) optionally, re-assessing the position of the balloon if the blood does not clear and repositioning the balloon if needed.

Those skilled in the art will recognize or be able to ascertain using no more than routine experimentation, many equivalents to the specific embodiments of the method and compositions described herein. Such equivalents are intended to be encompassed by the following claims.

1. A pediatric emergency kit comprising:
(i) an esophageal balloon catheter, comprising two ports in fluid communication with the proximal end of a catheter, wherein a first port is in fluid communication with the balloon and the second port is in fluid communication with the distal end of the catheter, and a flexible distal tip at the distal end of the catheter; and
(ii) an inflation hand pump, comprising a pressure gauge, wherein the pressure gauge includes colors and/or markings to distinguish pressures in the physiologic blood pressure range, optionally wherein the pressure gauge measures pressures in the range of 20 mmHg to 300 mmHg.

2. The pediatric emergency kit of claim 1, wherein the esophageal balloon catheter has a length of about 60 to about 90 cm.

3. The pediatric emergency kit of claim 1, wherein the balloon on the esophageal balloon catheter has a diameter in its widest region when fully inflated of about 25 mm to about 30 mm.

4. The pediatric emergency kit of claim 1, further comprising a guidewire that has a flexible distal tip, and wherein the guidewire has a suitable size and shape for the esophageal balloon catheter to fit over the guidewire, and to allow the esophageal balloon dilation catheter to be advanced along the guidewire.

5. The pediatric emergency kit of claim 1, wherein the esophageal balloon catheter is inside a first container or compartment that is sealed and/or locked, optionally with a break-away tab, optionally the kit further comprises (iii) a 50-70 cc syringe and (iv) a 3-way luer lock stopcock, wherein the 3-way luer stopcock is configured for attachment to the first port of the esophageal balloon catheter.

6. The pediatric emergency kit of claim 5, wherein the first container or compartment further includes (iii) and (iv) therein.

7. The pediatric emergency kit of claim 5, further comprising a second container or compartment, wherein the second container or compartment contains therein (i′) a second esophageal balloon catheter and (ii) the inflation hand pump; optionally the second container or compartment further contains (iii′) a second 50-70 cc syringe, and (iv′) a second 3-way luer lock stopcock,

8. The pediatric emergency kit of claim 7, wherein both the first and second containers or compartments are enclosed in a third container or compartment that is configured to be opened and closed more than one time for reuse.

9. The pediatric emergency kit of claim 1, further comprising instructions for use, optionally wherein one or more of the instructions are provided via a QR code.

10. The pediatric emergency kit of claim 1, wherein the maximum pressure denoted on the pressure gauge is in the range of about 300 mmHg to about 250 mmHg, 300 mmHg to 200 mmHg, about 250 mmHg to about 200 mmHg, or about 200 mmHg.

11. The pediatric emergency kit of claim 1, wherein the distal end of the esophageal balloon catheter includes an opening having a suitable size and shape for a guidewire to fit therein.

12. The pediatric emergency kit of claim 1, wherein the outer surface of the catheter in the esophageal balloon catheter includes two or more radiopaque markings, optionally wherein at least a portion of the radiopaque markings are located in the region of the catheter located within the balloon.

13. The pediatric emergency kit of claim 1, wherein the esophageal balloon catheter further comprises a third port in fluid communication with the proximal end of a catheter and with one or more additional ports on the side of the distal end of the catheter positioned between the distal end of the balloon and the flexible distal tip of the catheter.

14. The pediatric emergency kit of claim 1, further comprising a pressure relief valve configured to open when the pressure in the balloon reaches a set pressure, wherein the pressure relief valve is in in fluid communication with the outlet of the inflation bulb,
optionally the pressure relief valve opens automatically when the pressure in the balloon is in the range of about 150 mmHg to 200 mmHg.

15. The pediatric emergency kit of claim 1, wherein the pressure gauge dial is color coded, optionally wherein a first portion of the pressure gauge dial is a coded with a first color, such as green, and a second portion of the pressure gauge dial is coded with a second color, such as yellow, and a third portion of the pressure gauge dial is coded with a third color, such as red, optionally wherein the first portion of the pressure gauge dial ranges from 50 mmHg to 130 mmHg, and/or the second portion of the pressure gauge dial ranges from 130 mmHg to 150 mmHg.

16. A method of creating an esophageal tamponade in a pediatric patient in need thereof, comprising the steps of:
(a) inserting the flexible distal tip of the esophageal balloon catheter provided in the kit of claim 1 through the patient's mouth and advancing the flexible distal tip of the esophageal balloon catheter through the patient's esophagus until the balloon is aligned with the location of the arterio-esophageal fistula;
(b) confirming that the balloon of the esophageal balloon catheter is aligned with the location of the arterio-esophageal fistula;
(c) using the handpump provided in the kit of claim 1, to inflate the balloon to a pressure that is above systolic pressure, such as about 130 mmHg.

17. The method of claim 16, further comprising prior to step (a), (a′) inserting a guidewire through the patient's mouth, through the esophagus and into the patient's stomach, optionally wherein step (a′) further comprises inserting an endoscope through the patient's mouth through the esophagus and into the patient's stomach to facilitate positioning of the guidewire.

18. The method of claim 17, wherein step (a) further comprises inserting the proximal end of the guidewire into the opening at the distal end of the catheter and advancing the esophageal balloon dilation catheter over the guidewire until the balloon is aligned with the location of the arterio-esophageal fistula.

19. The method of claim 16, wherein the esophageal balloon catheter further comprises a third port in fluid communication with the proximal end of a catheter and with one or more additional ports on the side of the distal end of the catheter positioned between the distal end of the balloon and the flexible distal tip of the catheter, further comprising:
(d) aspirating with a syringe from the third port on the catheter to assess whether there is ongoing blood accumulation below the inflated balloon; and
if blood accumulation is observed:
(e) subsequently instilling saline through the third port,
(f) repeating syringe aspiration through the third port to observe if the accumulated blood has cleared, optionally repeating steps (d)-(f) until the accumulated blood has cleared, and
(g) optionally, re-assessing the position of the balloon if the blood does not clear and repositioning the balloon if needed.
